(19) United States
(12) Patent Application Publication
(10) Pub. No.: US 2024/0010610 A1
(43) Pub. Date: Jan. 11, 2024

(54) COMPOUNDS FOR TREATING SEGMENTAL PROGEROID SYNDROMES

(71) Applicants: Progelife, Marseille (FR); Université de Montpellier, Montpellier (FR); Centre National de la Recherche Scientifique, Paris (FR); École Nationale Supérieure de Chimie de Montpellier, Montpellier (FR)

(72) Inventors: Pierre CAU, Aix en Provence (FR); Eric DESSAUD, Marseille (FR); Muriel AMBLARD-CAUSSIL, Saint Vincent de Barbeyrargues (FR); Pascal VERDIE, Sainte Croix de Quintillargues (FR); Gilles SUBRA, Saint Gély Du Fesc (FR); Alexandre DEFOUX, Montpellier (FR); Claire NAVARRO, Martigues (FR); Sophie PERRIN, Marseille (FR)

(21) Appl. No.: 18/254,315

(22) PCT Filed: Nov. 24, 2021
(86) PCT No.: PCT/EP2021/082873

§ 371 Date: May 24, 2023

(30) Foreign Application Priority Data
Nov. 24, 2020 (EP) 20306430.8
Nov. 24, 2020 (EP) 20306431.6

(51) Int. Cl. C07C 237/22; C07C 271/02; C07D 241/12; A61P 43/00

(52) U.S. Cl. CPC C07C 237/22; C07C 271/02; C07D 241/12; A61P 43/00

(57) ABSTRACT

The present invention relates to a compound of the following formula (I):

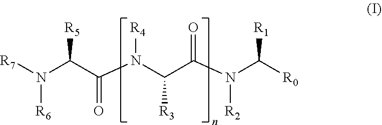

in particular for use in a method for preventing or treating a disease in an individual, more particularly for preventing or treating a disease is associated to progerin or to prelamin A.

### FIELD OF THE INVENTION

The present invention relates to novel compounds useful for treating segmental progeroid syndromes and to their use for treating such diseases.

### TECHNICAL BACKGROUND

Segmental progeroid syndromes are associated to mutations of proteins controlling the organization of the nuclear envelope as well as the organization and the functions of the nuclear matrix within the nucleoplasm. The proteins mutated in segmental progeroid syndromes are the LMNA-encoded lamins A/C, their protein partners within nuclear membrane, and proteins involved in the post-translational processing of LMNA gene products.

Among the segmental progeroid syndromes, progeria or Hutchinson-Gilford Progeria Syndrome (HGPS; OMIM #176670) is a rare genetic disorder which affects 1 in 4-8 million children with symptoms resembling normal adult ageing that include growth impairment, very thin skin, loss of subcutaneous fat, alopecia, osteoporosis, heart disease and atherosclerosis leading to shortened life span and death at about 13.5 years.

This syndrome is caused by a de novo missense point mutation c.1824 C>T within exon 11 of the LMNA gene that encodes lamin A. This mutation activates a cryptic donor splice site in exon 11 that leads to deletion of 50 amino acids at the carboxy-terminal globular domain resulting in a truncated protein lacking residues 607-656 of prelamin A, called progerin. Progerin, however, retains the C-terminal CAAX box, a target for farnesylation. Because an ZMPTE24 endoproteolytic cleavage site is lost, the truncated lamin/progerin is thus permanently farnesylated.

Lamins A/C, together with the B-type lamins, are the major components of the nuclear lamina, a fibrous network underlying the inner nuclear membrane. As such, at the cellular level, HGPS premature ageing disorder is characterized by dramatic defects in nuclear envelope structure, large-scale alterations in nuclear shape, blebbing, “herniations”, loss of some inner nuclear membrane (INM) proteins from one pole of the nucleus and disruption of the underlying heterochromatin. As lamin A is also a component of the internal nuclear matrix, its alteration in HGPS patient cells might affect the distribution and/or the structural organization of nuclear functional areas such as nucleoli, speckles and nuclear bodies. Abnormalities in nuclear matrix composition also result in defects in DNA and RNA metabolism steps, from DNA repair, leading to genome instability, to RNA transcription and splicing. Nuclear metabolic defects as well as their consequences on cell cycle, metabolic pathways and cell compartment functions lead to cellular senescence.

Other LMNA mutations affecting prelamin A maturation result in HGPS-like progeroid syndromes, which severity depends essentially on the quantities of progerin/prelamin A isoforms produced (Barthélémy et al. (2015). Eur J Hum Genet 23(8): 1051-1061). Two other syndromes, restrictive dermopathy (RD), a perinatal lethal genodermatosis, and type B mandibuloacral dysplasia (MAD-B), a relatively milder progeroid syndrome, have also been associated to pathological accumulation of prelamin A, mostly resulting from mutations in ZMPSTE24. Furthermore, several atypical progeroid syndromes (APS) or atypical Werner syndrome (AWS) with clinical features overlapping with HGPS and other prelamin A-linked disorders have been associated to missense mutations in the LMNA gene (Grelet et al. (2019). Orphanet Journal of Rare Diseases 14(1): 288). Besides, Nestor-Guillermo progeria syndrome is another progeroid disease caused by a mutation in BANF1 encoding BAF, a nuclear protein partner of lamin A and of emerin and linking chromatin to nuclear envelope (Cabanillas et al. (2011). Am J Med Genet A 155A: 2617-2625).

Recently, the farnesyl transferase inhibitor (FTI) lonafarnib has been approved by the US Federal Drug Administration (FDA) for reducing the risk of death due to Hutchinson-Gilford progeria syndrome and for the treatment of certain processing-deficient progeroid laminopathies. Indeed, lonafarnib treatment has been shown to be associated to a reduced mortality rate in progeria patients (Gordon et al. (2018) JAMA 319: 1687-1695). However, some aspects of the disease, such as insulin resistance, lipodystrophy, joint contractures and skin are not improved by the treatment (Gordon et al. (2012) Proc. Natl. Acad. Sci. USA 109:16666-16671).

Accordingly, there is still a need for alternative treatments for segmental progeroid syndromes.

### SUMMARY OF THE INVENTION

The inventors have now synthesized novel compounds which are effective at decreasing progerin levels in cells of HPGS individuals.

Accordingly, the present invention relates to a compound of the following formula (I):

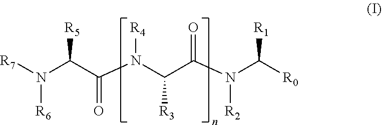

wherein:
n represents: 0, 1 or 2;
R₀ represents an aldehyde group or a protected aldehyde group;
R₂, R₄, and R₆, identical or different, with the proviso that when n=2 the two Ra groups may be identical or different, represent: H (hydrogen atom); an alkyl group having from 1 to 6 carbon atoms, optionally substituted by one or more amino groups or carboxylic acid groups; or an alkaryl or aryl group having from 5 to 10 carbon atoms, optionally substituted by one or more amino groups, hydroxyl group or carboxylic acid groups; in particular an isobutyl group, an isopentyl group, a phenylethyl group or an hydroxyphenylethyl group;
R₁, R₃ and R₅, identical or different, with the proviso that when n=2 the two R₃ groups may be identical or different, represent: H (hydrogen atom), an Arginine (Arg, R) functional group, a Leucine (Leu, L) functional group, a Norleucine (Nle) functional group, a Methionine (Met, M) functional group, a Phenylalanine (Phe, F) functional group, a Valine (Val, V) functional group, a Norvaline (Nva) functional group, or a Tyrosine (Tyr, Y) functional group; and
R₇ represents:
   a protecting group, or
   a group of the following formula (II):

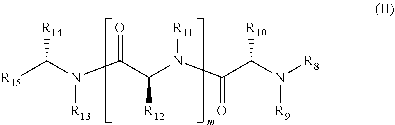

wherein:
   m represents: 0, 1 or 2;
   R₉, R₁₁ and R₁₃, identical or different, with the proviso that when n=2 the two R₁₁ groups may be identical or different, represent: H (hydrogen atom); an alkyl group having from 1 to 6 carbon atoms, optionally substituted by one or more amino group or carboxylic acid group; or an alkaryl or aryl group having from 5 to 10 carbon atoms, optionally substituted by one or more amino groups, hydroxyl groups or carboxylic acid groups; in particular an isobutyl group, an isopentyl group, a phenylethyl group or an hydroxyphenylethyl group;
   R₁₀, R₁₂ and R₁₄, identical or different, with the proviso that when n=2 the two R₁₂ groups may be identical or different, represent: H (hydrogen atom), an Arginine (Arg, R) functional group, a Leucine (Leu, L) functional group, a Norleucine (Nle) functional group, a Methionine (Met, M) functional group, a Phenylalanine (Phe, F) functional group, a Valine (Val, V) functional group, a Norvaline (Nva) functional group, or a Tyrosine (Tyr, Y) functional group;
   R₈ represents a linking moiety; and
   R₁₅ represents an aldehyde group or a protected aldehyde group, or a pharmaceutically acceptable salt thereof.

In a preferred embodiment, the present invention relates to a compound of formula (I) as defined above provided that when n=1, R₀ is an aldehyde group, R₂, R₄ and R₆ all represent H (hydrogen atom), R₇ is a Z protecting group, and R₃ and R₅ both represent a leucine functional group, then R₁ does not represent a Leucine, a Norvaline or a Phenylalanine functional group.

In another preferred embodiment, the present invention relates to a compound of formula (I) as defined above, provided it is different from:

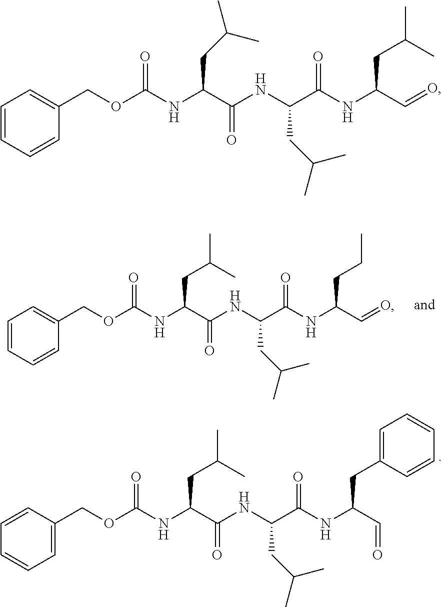 [SMILES: CC(C)C[C@@H](C=O)NC(=O)[C@H](CC(C)C)NC(=O)[C@H](CC(C)C)NC(=O)OCc1ccccc1 ; CCC[C@@H](C=O)NC(=O)[C@H](CC(C)C)NC(=O)[C@H](CC(C)C)NC(=O)OCc1ccccc1 ; CC(C)C[C@H](NC(=O)OCc1ccccc1)C(=O)N[C@@H](CC(C)C)C(=O)N[C@H](C=O)Cc1ccccc1]

The present invention also relates to a compound of formula (I) as defined above, or a pharmaceutically acceptable salt thereof, for use as a medicament or in a method for preventing or treating a disease, in particular associated to progerin or to prelamin A in an individual.

The present invention also relates to the use of a compound of formula (I) as defined above, or a pharmaceutical acceptable salt thereof, for the manufacture of a medicament intended for preventing or treating a disease, in particular associated to progerin or to prelamin A in an individual.

The present invention also relates to a pharmaceutical composition comprising as active ingredient a compound of formula (I) as defined above, or a pharmaceutically acceptable salt thereof, optionally in association with at least one pharmaceutically acceptable carrier or excipient, preferably for use in a method for preventing or treating a disease, in particular associated to progerin or to prelamin A in an individual.

The present invention also relates to a method for the prevention or treatment of a disease, in particular associated to progerin or to prelamin A in an individual, comprising administering to the individual an effective quantity of a compound of formula (I) as defined above, or a pharmaceutically acceptable salt thereof, or of a pharmaceutical composition as defined above.

### DESCRIPTION OF THE INVENTION

As intended herein, the word “comprising” is synonymous to “include” or “contain”. When a subject-matter is said to comprise one or several features, it is meant that other features than those mentioned can be comprised in the subject-matter. Conversely, the expression “constituted of” is synonymous to “consisting of”. When a subject-matter is said to consist of one or several features, it is meant that no other features than those mentioned are comprised in the subject-matter.

### Compound

Compounds of formula (I) can be readily synthesized by one of skill in the art, in particular by solid phase peptide synthesis (SPPS).

Protecting groups for protecting the N-terminus of peptides are well known to one of skill in the art and any such protecting group can be used according to the invention. However, it is preferred that the protecting group according to the invention is selected from the group consisting of carboxybenzyl (Z or Cbz), acetyl (Ac), dichlorobenzyl, pyrazinyl carbonyl, difluorophenyl.

As intended herein, an aldehyde group is a group of the following formula:

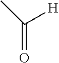

Protected aldehyde groups are well known to one of skill in the art and any such protected aldehyde group can be used according to the invention. However, it is preferred that the protected aldehyde group according to the invention is selected from the group consisting of an amide, a carboxylic acid, a semicarbazone, an imine, an oxyme, an hydrazone, a sodium bisulfite and a thiazolidine. More preferably, the protected aldehyde group according to the invention is selected from the groups represented by the following formulae:

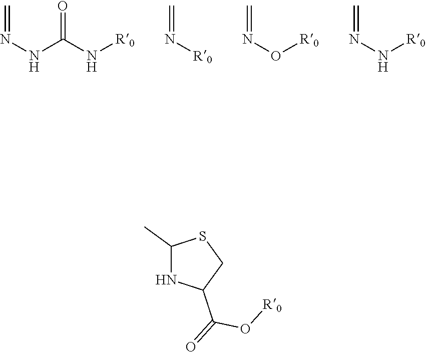

wherein R′₀ represents H (hydrogen atom) or a group comprising from 1 to 100 carbon atoms, preferably from 1 to 50 carbon atoms and more preferably from 1 to 20 carbon atoms. Where R′₀ represents a group comprising from 1 to 20, 50 or 100 carbon atoms, it is preferably a polar group or a polymer ligation group. Most preferably, the protected aldehyde group according to the invention is represented by the following formula:

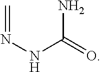

As intended herein, a linking moiety refers to any group capable of bridging two amine groups. Preferably, the linking moiety has from 3 to 20 carbon atoms and comprises at least 2 carboxylic acid groups. As will clear to one of skill in the art the two carboxylic acid groups of the preferred liking moiety form amide bonds with the two amine groups the linking moiety is bridging. More preferably, the linking moiety, when bridging the two amine groups is selected from the groups having the following formulae:

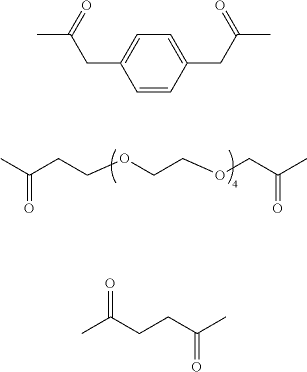

As should be clear to one of skill in the art, when R₇ represents a group of formula (II), the compound of formula (I) can be represented by the following formula (III):

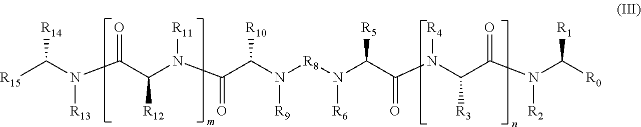

Preferably, the compound of formula (I) according to the invention is selected from the group consisting of:

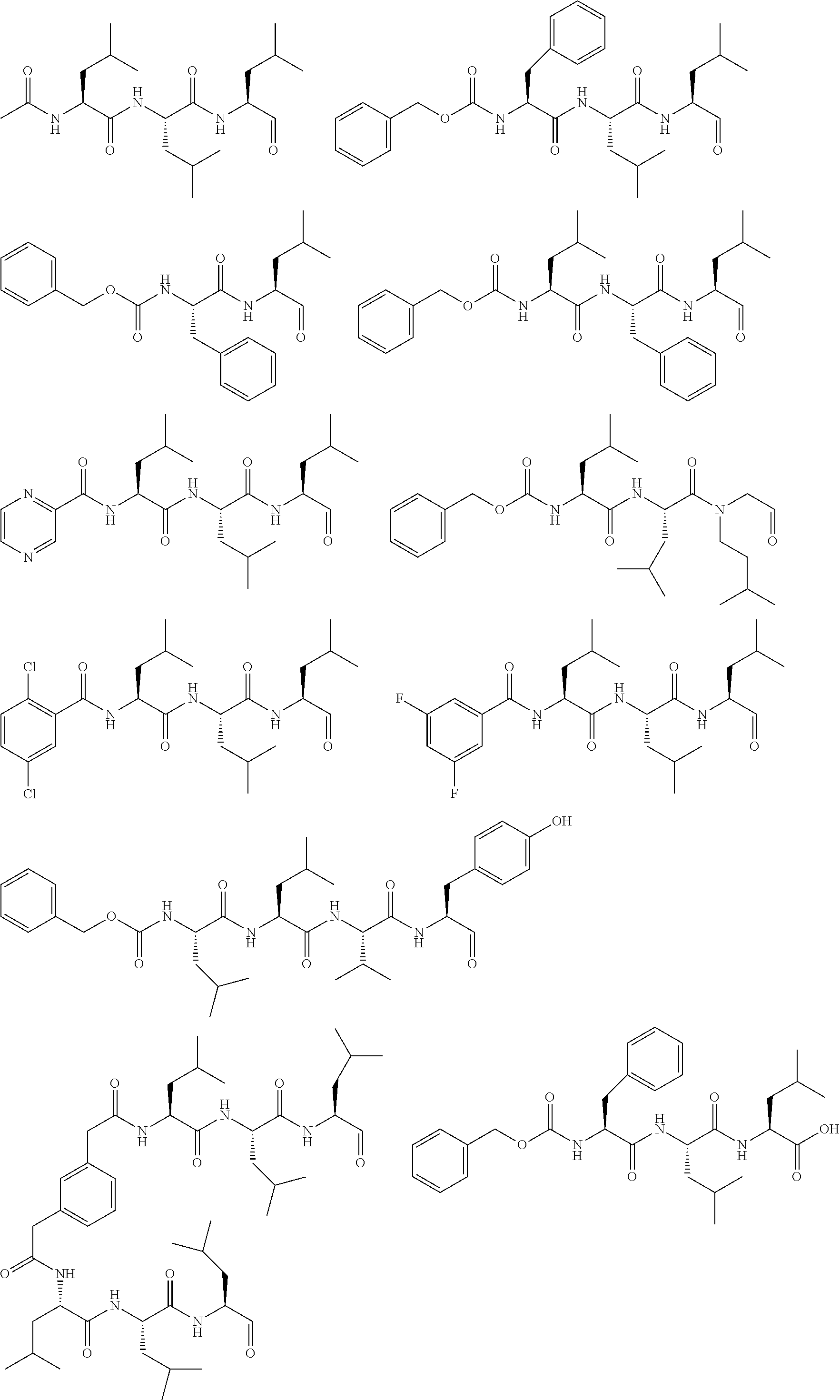

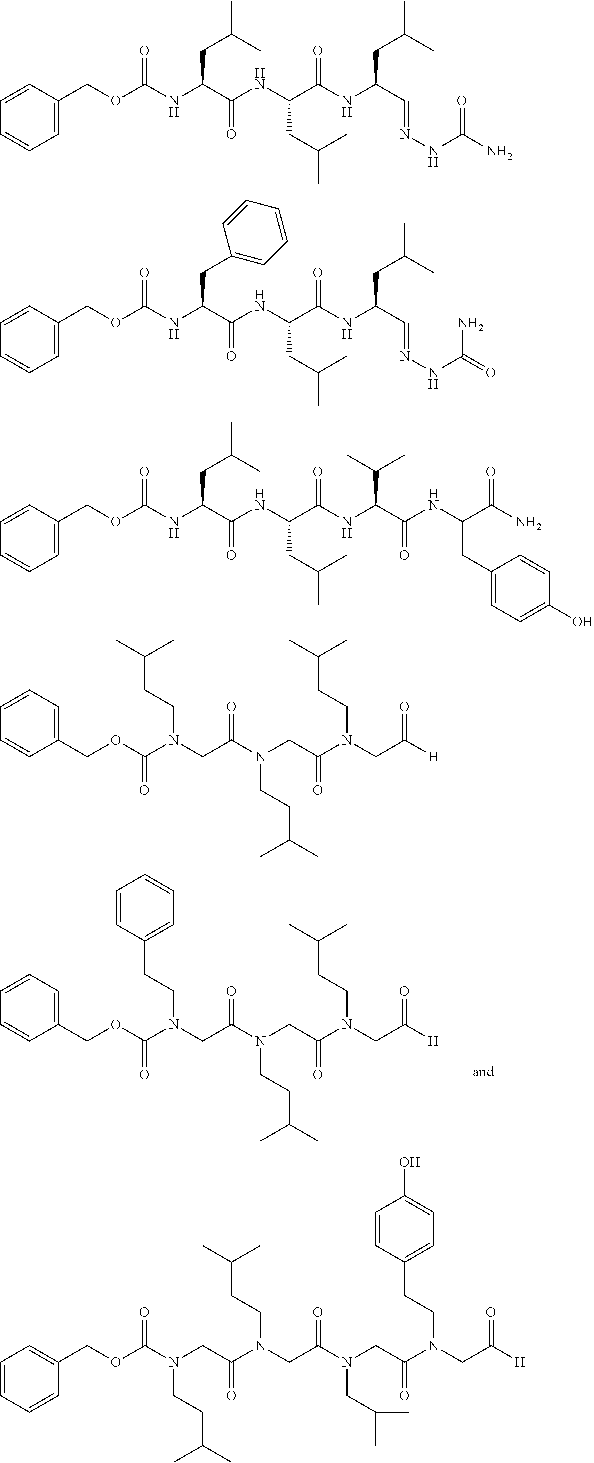

### Disease

As intended herein, a disease associated to prelamin A or to progerin relates to a disease caused by a cellular accumulation of prelamin A or of progerin, i.e. a farnesylated truncated form of prelamin A, in particular lacking residues 607-656.

Preferably, the disease according to the invention is a segmental progeroid syndrome, in particular associated with LMNA-encoded lamins A/C, more preferably selected from the group consisting of progeria, in particular Hutchinson-Gilford Progeria Syndrome (HGPS), an HGPS-like syndrome, restrictive dermopathy, mandibuloacral dysplasia type B, an atypical progeroid syndrome, an atypical Werner syndrome, and Nestor-Guillermo progeria syndrome. More preferably, the disease according to the invention is progeria or Hutchinson-Gilford Progeria Syndrome (HGPS).

### Treatment

Preferably, the method according to the invention decreases the individual's cellular concentration of progerin or of prelamin A.

A “decrease” is defined by reference to the situation before the method according to the invention is applied to the individual. Numerous methods for determining the cellular concentration of a protein are known to one of skill in the art. By way of example, one can perform an ELISA assay on cellular extracts of a biopsy which has been obtained from the individual. Indirectly, it is also possible to determine the concentration of mRNAs encoding the protein by a quantitative RT-PCR.

### Individual

Preferably, the individual as defined above is a human. Preferably, the individual as defined above is at risk or afflicted with a disease associated to progerin or prelamin A, in particular progeria or HGPS.

### Pharmaceutical Composition

The compound of formula (I) according to the invention or a pharmaceutically acceptable salt thereof can be comprised in a pharmaceutical composition which can comprise at least one pharmaceutically acceptable vehicle or excipient. The pharmaceutically acceptable vehicle or excipient can be selected from dispersants, solubilizers, nebulizers, stabilizers, preservatives, etc. Besides, pharmaceutically acceptable vehicle or excipient which can be used in formulations, in particular liquid and/or injectable formulations, are preferably selected from sucrose, lactose, starch, methylcellulose, hydroxymethylcellulose, carboxymethylcellulose, croscarmellose sodium, lactose monohydrate, magnesium stearate, microcrystalline cellulose, povidone, sodium lauryl sulfate, mannitol, gelatin, lactose, vegetable oils, acacia gum, liposomes, etc.

### Administration

The compound of formula (I) or a pharmaceutically acceptable salt thereof or the pharmaceutical composition as defined above can be administered orally, parenterally, mucosally or cutaneously. The parenteral route preferably comprises subcutaneous, intravenous, intramuscular or intraperitoneal administration, although the latter is rather reserved for animals. The mucosal route preferably comprises nasal administration, oro-pharyngeal administration, pulmonary administration or administration via the rectal mucosa. The cutaneous route advantageously comprises the dermal route, in particular via a transdermal device, typically a patch.

The compound of formula (I) according to the invention or a pharmaceutically acceptable salt thereof or the pharmaceutical composition as defined above can be formulated in the form of injectable solutions or suspensions, gels, oils, tablets, suppositories, powders, gel capsules, capsules, aerosols, etc., optionally by means of galenical forms or of devices which provide sustained and/or delayed release. For this type of formulation, an agent such as cellulose, carbonates, starches, or approved biopolymers (e.g. PEG, chitosan, hyaluronic acid polymers) is advantageously used. Galenic forms made of biopolymer-drug conjugates can be encapsulated in patient red blood cells that are further intravenously injected after encapsulation, thus allowing such sustained and/or delayed drug release.

The compound of formula (I) according to the invention or a pharmaceutically acceptable salt thereof or the pharmaceutical composition as defined above can be administered to the individual as defined above at a dose between 0.1 mg and 1000 mg, preferably between 0.1 mg and 100 mg, more preferably between 1 mg and 100 mg, of the compound of formula (I) or a pharmaceutically acceptable salt thereof as defined above. Of course, those skilled in the art are able to adjust the dose of the compound of formula (I) or a pharmaceutically acceptable salt thereof as defined above according to the weight or body surface area of the individual to be treated. Preferably, the dosage range of the compound of formula (I) according to the invention or a pharmaceutically acceptable salt thereof is from 0.1 mg/day and 1000 mg/day, preferably between 0.1 mg/day and 100 mg/day, more preferably between 1 mg/day and 100 mg/day.

The invention will be further specified by the following non-limiting Examples.

### EXAMPLES

### Examples 1-4

The following compounds were synthesized:

| Compound | Structure |
|---|---|
| 3 | 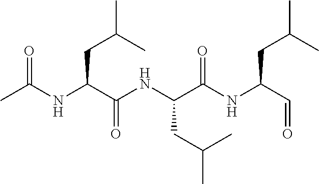 [SMILES: CC(=O)N[C@@H](CC(C)C)C(=O)N[C@@H](CC(C)C)C(=O)N[C@H](C=O)CC(C)C] |
| 4 | 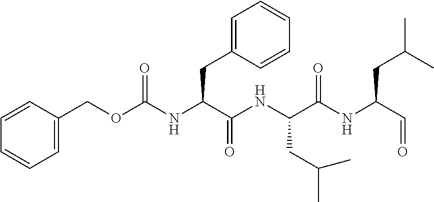 [SMILES: CC(C)C[C@@H](C=O)NC(=O)[C@H](CC(C)C)NC(=O)[C@H](Cc1ccccc1)NC(=O)OCc1ccccc1] |
| 6 | 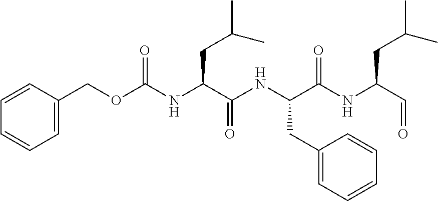 [SMILES: CC(C)C[C@@H](C=O)NC(=O)[C@H](Cc1ccccc1)NC(=O)[C@H](CC(C)C)NC(=O)OCc1ccccc1] |
| 7 | 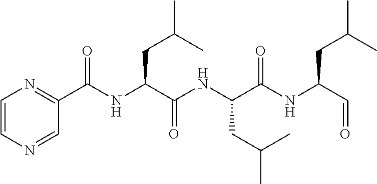 [SMILES: CC(C)C[C@@H](C=O)NC(=O)[C@H](CC(C)C)NC(=O)[C@H](CC(C)C)NC(=O)c1cnccn1] |
| 8 | 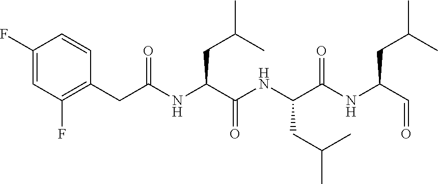 [SMILES: CC(C)C[C@@H](C=O)NC(=O)[C@H](CC(C)C)NC(=O)[C@H](CC(C)C)NC(=O)Cc1ccc(F)cc1F] |
| 10 | 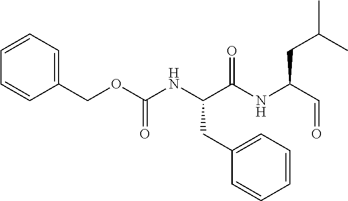 [SMILES: CC(C)C[C@@H](C=O)NC(=O)[C@H](Cc1ccccc1)NC(=O)OCc1ccccc1] |
| 11 | 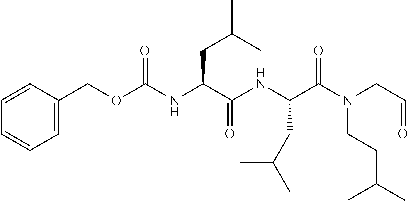 [SMILES: CC(C)CCN(CC=O)C(=O)[C@H](CC(C)C)NC(=O)[C@H](CC(C)C)NC(=O)OCc1ccccc1] |
| 12 | 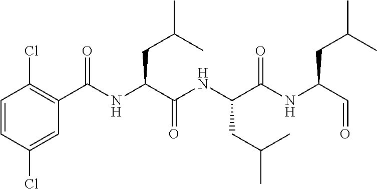 [SMILES: CC(C)C[C@@H](C=O)NC(=O)[C@H](CC(C)C)NC(=O)[C@H](CC(C)C)NC(=O)c1cc(Cl)ccc1Cl] |
| 13 | 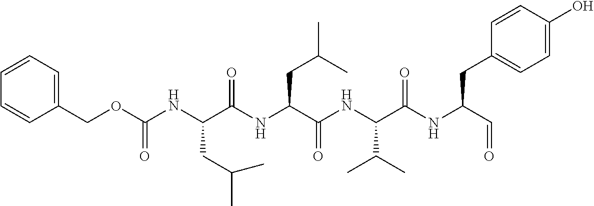 [SMILES: CC(C)C[C@H](NC(=O)OCc1ccccc1)C(=O)N[C@@H](CC(C)C)C(=O)N[C@H](C(=O)N[C@H](C=O)Cc1ccc(O)cc1)C(C)C] |
| 14 | 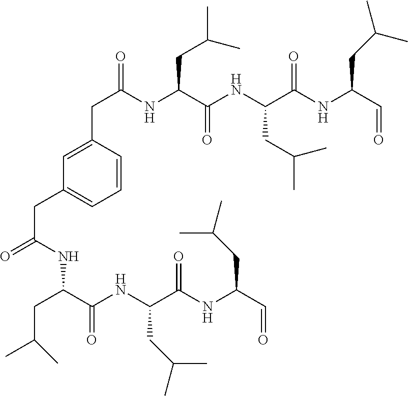 [SMILES: CC(C)C[C@@H](C=O)NC(=O)[C@H](CC(C)C)NC(=O)[C@H](CC(C)C)NC(=O)Cc1cccc(CC(=O)N[C@@H](CC(C)C)C(=O)N[C@@H](CC(C)C)C(=O)N[C@H](C=O)CC(C)C)c1] |
| 15 | 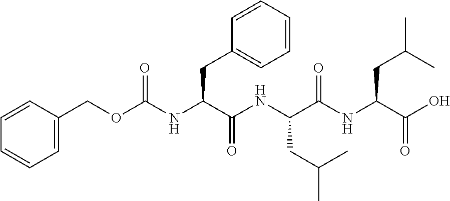 [SMILES: CC(C)C[C@H](NC(=O)[C@H](CC(C)C)NC(=O)[C@H](Cc1ccccc1)NC(=O)OCc1ccccc1)C(=O)O] |
| 16 | 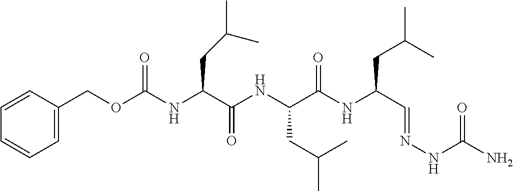 [SMILES: CC(C)C[C@@H](/C=N/NC(N)=O)NC(=O)[C@H](CC(C)C)NC(=O)[C@H](CC(C)C)NC(=O)OCc1ccccc1] |
| 17 | 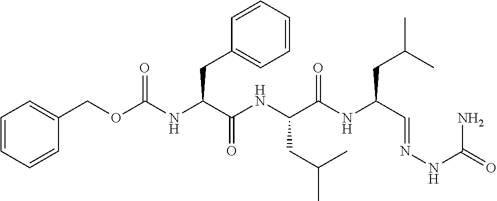 [SMILES: CC(C)C[C@@H](/C=N/NC(N)=O)NC(=O)[C@H](CC(C)C)NC(=O)[C@H](Cc1ccccc1)NC(=O)OCc1ccccc1] |
| 18 | 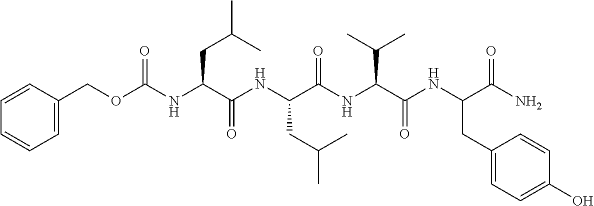 [SMILES: CC(C)C[C@H](NC(=O)OCc1ccccc1)C(=O)N[C@@H](CC(C)C)C(=O)N[C@H](C(=O)NC(Cc1ccc(O)cc1)C(N)=O)C(C)C] |
| 19 | 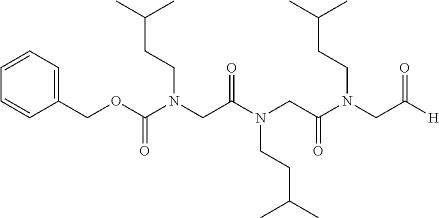 [SMILES: CC(C)CCN(CC=O)C(=O)CN(CCC(C)C)C(=O)CN(CCC(C)C)C(=O)OCc1ccccc1] |
| 20 | 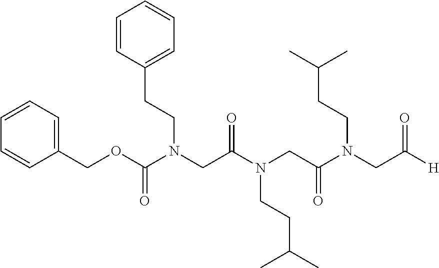 [SMILES: CC(C)CCN(CC=O)C(=O)CN(CCC(C)C)C(=O)CN(CCc1ccccc1)C(=O)OCc1ccccc1] |
| 21 | 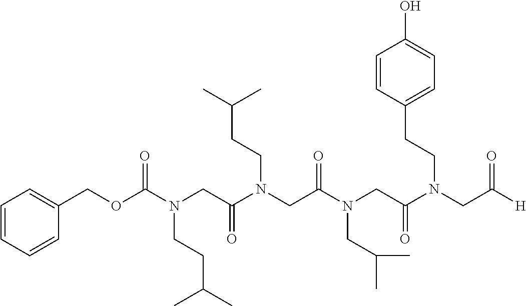 [SMILES: CC(C)CCN(CC(=O)N(CC(=O)N(CC=O)CCc1ccc(O)cc1)CC(C)C)C(=O)CN(CCC(C)C)C(=O)OCc1ccccc1] |

The synthesis of representative compounds is detailed below.

### General Methods

All peptidyl aldehydes were prepared by solid phase peptide synthesis (SPPS) using Weinreb amide bound linker (N-Fmoc-N-methoxy-3-aminopropionic acid). Reduction of this amide by lithium aluminum hybride leads to peptidyl aldehyde (Fehrentz et al., Tet. Let. 36, 143, 7871-7874 (1995)). Two different resins have been used: a commercial Weinreb AM resin purchased at Merck and a RAM amphisphere resin purchased at Agilent Technologies which was functionalized by synthetized Fmoc-N-methoxy-3-aminopropanoic acid before the SPPS steps. Peptidyl aldehydes with different N-protecting groups and amino acids were prepared as defined in examples 1 and 2. A peptide-peptoid hybrid was prepared according to the procedure given in example 3 and a dimer of peptidyl aldehydes was prepared according to Example 4. All the compounds were characterized by proton Nuclear Magnetic Resonance (NMR ¹H) and their purity were determined by high performance liquid chromatography (HPLC) coupled to mass spectrometry (MS).

All Fmoc-amino acid derivatives and (1-[Bis(dimethylamino)methylene]-1H-1,2,3-triazolo[4,5-b]pyridinium 3-oxide hexafluorophosphate (HATU) were purchased from Iris Biotech GmbH. Piperidine, N,N-diisopropylethylamine (DIEA), trifluoroacetic acid (TFA), triisopropylsilane (TIS), dichloromethane (DCM), N,N-dimethylformamide (DMF), acetonitrile, and 1,2-dichloroethane (DCE) were provided by Sigma Aldrich. Fmoc Rink amide AmphiSpheres™ 40 RAM 0.39 mmol/g 75-150 μm resin was purchased from Agilent Technologies. All solvents used for HPLC and LCMS were purchased from Sigma Aldrich in gradient grade or reagent quality. All final compounds were purified by reversed-phase HPLC and the purity assessed by LC-MS.

Samples for LC-MS analyses were prepared in acetonitrile/water mixture (50:50, v/v) containing 0.1% TFA. The LC-MS system consisted of a Waters Alliance 2695 HPLC coupled to a Water Micromass ZQ spectrometer (electrospray ionization mode, ESI+). All analyses were carried out using a Phenomenex Onyx reversed-phase column (25×4.6 mm). A flow rate of 3 mL/min and a gradient from 0 to 100% of B over 2.5 min were used. Eluent A: water/0.1% formic acid; eluent B: acetonitrile/0.1% formic acid. UV detection was performed at 214 nm. Positive ion electrospray mass spectra were acquired at a solvent flow rate of 100-500 μL/min. Nitrogen was used for both the nebulizing and drying gas. The data were obtained in a scan mode in 0.1 s intervals; 10 scans were summed up to get the final spectrum.

### Synthesis of Fmoc-N-methoxy-3-aminopropionic Acid

### Preparation of benzyl 3-amino-N-methoxy propanoate

3.34 g (40 mmol) of methoxylamine hydrochloride were dissolved in 100 ml of acetonitrile and 13 mL of DIEA (80 mmol). 3.04 mL of benzyl acrylate (20 mmol) were then added. The mixture was stirred for 20 h at reflux. Then, the solvent was concentrated under reduced pressure to dryness and ethyl acetate was added. The resulting organic solution was washed twice with water, once with a saturated solution of sodium chloride (NaCl), dried over MgSO₄ and concentrated under reduced pressure to afford 3.2 g of a slightly brown oil product with a crude yield of 76%.

### Preparation of benzyl 3-amino-N-Fmoc-N-methoxy-propanoate

To 3.2 g of benzyl 3-amino-N-methoxy propanoate (15 mmol) in water (20 mL) was added 2.085 mL of triethylamine (15 mmol, 1 eq.) and 3.48 g (13.5 mmol, 0.9 eq.) of Fmoc-Cl solubilized in 15 mL of dioxane. After 2 h stirring at room temperature, ethyl acetate (100 mL) was added and the resulting solution was washed twice with a solution of 0.1M HCl, saturated KHSO₄ and saturated NaCl, dried over MgSO₄ and concentrated under vacuum. 4.88 g (11.3 mmol) were obtained as a slightly brown oil (crude yield: 75%) and used without further treatment for the preparation of the following compound.

### Preparation of 3-amino-N-Fmoc-N-methoxy-propanoic acid

4.88 g of 3-amino-N-Fmoc-N-methoxy-propanoate (11.3 mmol) was hydrogenated for 28 h at room temperature in EtOH (50 mL) in the presence of a 10% Pd/C catalyst. The catalyst was then removed by filtration on celite and the resulting solution was concentrated under vacuum to give 3.62 g (10.61 mmol, crude yield: 94%) of a slightly brown oil. The product was then purified by flash chromatography (dichloromethane and methanol as mobile phase) to obtain 1.8 grams (5.3 mmol) of pure 3-amino-N-Fmoc-N-methoxy propionic acid (final yield: 25%).

### Example 1: Preparation of Compound 6 (Z-Leucine-Phenylalanine-Leucinal) on Weinreb Linker Functionalized Amphisphere RAM Resin

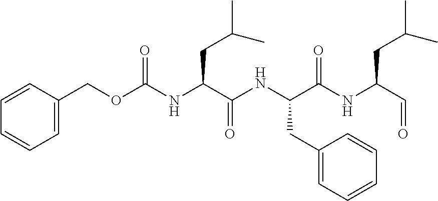 [SMILES: CC(C)C[C@@H](C=O)NC(=O)[C@H](Cc1ccccc1)NC(=O)[C@H](CC(C)C)NC(=O)OCc1ccccc1]

256 mg (0.1 mmol) of Amphisphere RAM resin (0.39 mmol/g·75-150 μm) were swollen in dichloromethane (DCM) for 15 min. The amine of the resin was then deprotected from its Fmoc group in a Pip/DMF 20% mixture (2×5 min). The resin was washed with dimethylformamide (DMF) and DCM.

160 mg of 3-amino-N-Fmoc-N-methoxy-propanoic acid (0.5 mmol, 5 eq.) were solubilized in 2 mL of DMF and added to the syringe containing the resin. 170 μL of DIPEA (10 eq.) and 1 mL of 0.5M HATU (0.5 mmol, 5 eq.) were added. The reaction was stirred for 1 hour at room temperature. After completion of the reaction monitored by the Kaiser test (detection of primary amine), the resin was washed with DMF and DCM. Then the Fmoc protecting group was removed by treatment of the resin with 20% piperidine in DMF (2×2.5 min) and the resin was washed with DMF and DCM.

The peptide was then elongated on the resin by Fmoc SPPS using HATU/DIPEA as coupling reagent. The amino acid couplings were performed using amino acid solution (0.5 M), DIPEA and HATU solution (0.5 M). 1 mL of amino acid solution (0.5 mmol, 5 eq.) was first introduced following by 180 μL of DIPEA (1 mmol, 10 eq.) and 1 mL of HATU 0.5 M solution (0.5 mmol, 5 eq.). The coupling reaction was performed two times, followed by the deprotection of the Fmoc group by a Pip/DMF 20% mixture.

| | | | DIEA | | Pip/DMF | | |
|---|---|---|---|---|---|---|---|
| AA | | AA 0.5M | (μL) | HATU 0.5M | 20% | Repetition | |
| Fmoc-Leu-OH | Coupling | 1 mL | 180 | 1 mL | — | 2 × 10 | min |
| | | (0.5 mmol, | | (0.5 mmol, | | | |
| | | 5 eq.) | | 5 eq.) | | | |
| | Deprotection | — | — | — | 5 mL | 2 × 2.5 | min |
| Fmoc-Phe-OH | Coupling | 1 mL | 180 | 1 mL | — | 2 × 5 | min |
| | | (0.5 mmol, | | (0.5 mmol, | | | |
| | | 5 eq.) | | 5 eq.) | | | |
| | Deprotection | — | — | — | 5 mL | 2 × 2.5 | min |
| Z-Leu-OH | Coupling | 1 mL | 180 | 1 mL | — | 2 × 5 | min |
| | | (0.5 mmol, | | (0.5 mmol, | | | |
| | | 5 eq.) | | 5 eq.) | | | |

The resin was then washed by DMF and DCM and dried.

The resin was then swollen in 20 mL of anhydrous tetrahydrofuran (THF) for 15 min at 0° C. under moderate stirring and argon bubbling. 800 μL of commercial LiAlH₄ 1M (8 eq.) in anhydrous THF were slowly introduced and the reaction was stirred at 0° C. for 45 min, then quenched with 30 mL of an aqueous solution of KHSO₄ 5% and left under stirring for 15 min. The suspension was then filtered and washed with DCM. The peptide aldehyde was then extracted from the aqueous solution with DCM (4 times). The combined organic phases were then washed one time with saturated NaCl solution and dried over MgSO₄. The solution was filtrated and the organic solvent was evaporated in vacuo. 45 mg of product were obtained. The Z-Leu-Phe-Leucinal peptide was purified by preparative reversed phase liquid chromatography using acetonitrile/water 0.1% trifluoroacetic acid as mobile phase. 25.5 mg Z-Leu-Phe-Leucinal were obtained (yield: 50%) with a purity of 100%.

### Example 2: Preparation of Compound 4 (Z-Phenylalanine-Leucine-Leucinal) on Commercial Weinreb Resin

370 mg (0.2 mmol) of commercial Weinreb resin (0.54 mmol/g, 100-200 μm) were swollen in DCM for 15 min. The Fmoc protecting group was removed by treatment of the resin with 20% piperidine in DMF (2×2.5 min) and the resin was washed with DMF and DCM.

The peptide elongation was performed as described above:

| | | | DIEA | | Pip/DMF | | |
|---|---|---|---|---|---|---|---|
| AA | | AA 0.5M | (μL) | HATU 0.5M | 20% | Repetition | |
| Fmoc-Leu-OH | Coupling | 2 mL | 320 | 2 mL | — | 2 × 10 | min |
| | | (1.0 mmol, | | (1.0 mmol, | | | |
| | | 5 eq.) | | 5 eq.) | | | |
| | Deprotection | — | — | — | 5 mL | 2 × 2.5 | min |
| Fmoc-Leu-OH | Coupling | 2 mL | 320 | 2 mL | — | 2 × 10 | min |
| | | (1.0 mmol, | | (1.0 mmol, | | | |
| | | 5 eq.) | | 5 eq.) | | | |
| | Deprotection | — | — | — | 5 mL | 2 × 2.5 | min |
| Z-Phe-OH | Coupling | 2 mL | 320 | 2 mL | — | 2 × 10 | min |
| | | (1.0 mmol, | | (1.0 mmol, | | | |
| | | 5 eq.) | | 5 eq.) | | | |

The resin was then swollen in 15 mL of anhydrous THF for 15 min at 0° C. under moderate stirring and argon bubbling. 1 mL (5 eq.) of commercial LiAlH₄ 1M in anhydrous THF was slowly introduced and the reaction was left under stirring at 0° C. for 30 min, then quenched with few drops of aqueous solution of KHSO₄ 1 M and stirred for min. The suspension was then filtered and washed with THF and ethyl acetate. The solution was diluted with ethyl acetate and washed with an aqueous solution of KHSO₄ 5%, NaHCO₃ 1M and saturated NaCl then dried over MgSO₄. The solution was filtrated and the organic solvent was evaporated in vacuo. 61 mg of crude compound were obtained and purified by preparative reversed phase liquid chromatography using acetonitrile/water 0.1% trifluoroacetic acid as mobile phase to yield to 18.16 mg of the title aldehyde Z-Phe-Leu-Leucinal (yield: 18%) with a purity of 100%.

### Example 3: Preparation of Compound 11 (Z-Leu-Leu-N(Isopentylamine)Glycinal) on Commercial Weinreb Resin

252 mg (0.2 mmol) of commercial Weinreb resin (0.62 mmol/g, 75-150 μm) were swollen in DCM for 15 min. The Fmoc protecting group was removed by treatment of the resin with 20% piperidine in DMF (2×2.5 min) and the resin was washed with DMF and DCM. The reaction was monitored by a chloranil test.

278 mg (2 mmol, 10 eq.) of bromoacetic acid were solubilized in 2 mL of DMF and introduced in the syringe containing the resin. 208 μL of N,N′-Diisopropylcarbodiimide (DIC) (2 mmol, 10 eq.) and 1 mL of 4-Dimethylaminopyridine (DMA P) 0.2M (1 eq.) were added and the suspension was left under stirring for 1 h. The reaction was repeat a second time in the same conditions. The resin was washed with DMSO, DMF and DCM then a chloranil test allowed to verify if the secondary methoxylamine was acylated.

2 mL of isopentylamine 1.5M (1.0 mmol, 5 eq.) in DMSO were added to the resin and the reaction was stirred overnight at room temperature. The resin was then washed with DMF and DCM and the presence of secondary amine was monitored by a chloranil test.

1 mL of Boc-Leu-OH·H₂O 1M (1.0 mmol, 5 eq.) in DMF were added in the syringe followed by 320 μL of DIPEA (10 eq.) and 2 mL of HATU 0.5M (1.0 mmol, 5 eq.). The reaction was stirred for 1 h 30. The coupling was repeat a second time in the same conditions. The resin was washed with DMF and DCM and a Kaiser test was performed to monitor the completion of the reaction.

The Boc protection was removed by treatment of the resin with 4 mL of TFA/DCM (1/1) for 90 min. The resin was then washed with isopropanol, DMF and DCM.

Z-Leu-OH 0.5M (5 eq.) in DMF (2 mL) was reacted with 208 μL of DIC for 15 min. Then, 2 mL of an oxyma pure solution 0.5M was added and the reaction mixture was stirred for 15 more minutes to generate the corresponding active ester. The preactivated Z-Leu-OH was then added to the resin and the reaction was left overnight. The resin was then washed with DMF and DCM and a Kaiser test monitored the acylation of the free amino group.

The Weinreb amide reduction was performed as described previously with 5 eq. of LiAlH₄. After treatment, 59 mg of crude compound as an oil were obtained.

After purification by preparative HPLC, 1.8 mg of pure Z-Leu-Leu-N(isopentylamine)glycinal were obtained (yield: 2%).

### Example 4: Preparation of Compound 14 (Dimer)

The peptidyl Fmoc-Leu-Leu-Leu-resin was prepared on a Weinreb linker functionalized RAM amphisphere resin by conventional SPPS as described in example 1.

Then, 0.8 g of the peptidyl resin (0.2 mmol peptidic equivalents) was treated by Pip/DMF 20:80 mixture 2×5 minutes for Fmoc removal. The resin was then treated by a TFA/DCM 50:50 mixture (6 mL) for 1 h 30. The suspension was filtrated and washed 3 times with DCM. The organic solution was evaporated in vacuo and the residue was precipitated in ether and centrifuged. The ether was removed and 86 mg of crude compound were recovered (0.18 mmol). The 0.18 mmol of H-Leu-Leu-Leu-Weinreb amide was solubilized in 2 mL of DMF. Then, 16 mg of m-phenylacetic acid (0.08 mmol, 0.45 eq.), 160 μL of DIEPA (5 eq.) and 0.9 mL of HATU 0.5M in DMF (2.5 eq.) were added and the solution was stirred for 24 h.

The solution was then diluted in water and the product was extracted with DCM. The organic phase was dried over MgSO₄ and evaporated in vacuo. The product was then purified by preparative HPLC and lyophilized to obtain 22 mg of the title dimer.

The 22 mg (0.002 mmol) of the purified product were solubilized in 10 mL of anhydrous THF under argon bubbling and stirred for 15 minutes. 0.64 mL (16 eq.) of commercial LiAlH₄ 1M in anhydrous THF were slowly introduced and the reaction was left under stirring at 0° C. for 1 h then quenched with few drops of aqueous solution of KHSO₄ 1M and left under stirring for 15 min. The solution was diluted in DCM and the organic phase was washed with KHSO₄ 5%, saturated NaHCO₃ and saturated NaCl aqueous solutions, dried over MgSO₄ and evaporated in vacuo. 43 mg of product were obtained and purified by preparative HPLC to obtain 4.13 mg (yield=5%) of the desired dimeric peptide with 90% of purity.

### Example 5: Progerin-Decreasing Effect

### Material and Methods

### Fibroblast Culture and Treatment

Fibroblasts from HGPS donors from passage 18 to 24 were cultured in DMEM low glucose (Life Technologies, Courtabœuf, France) containing 15% FBS (Life Technologies), 2 mM L-glutamine (Life Technologies) and 100 U/mL penicillin-streptomycin (Life Technologies) at 37° C. in a humidified atmosphere containing 5% CO2.

Fibroblasts were cultured in the presence of compounds according to the invention 3, 4, 6, 7, 8, 12, 13, 14 and 16 for 72 hours with renewal after 48 hours. Comparative compound 1 (Z-Leu-Leu-Leucinal) was added as a control. All molecules were diluted in DMSO at 10 mM. Concentrations from 100 μM to 0.0001 nM were used to test cell viability in 96-well plates. Drug efficiency was evaluated in 6-well plates.

### Cell Viability and Assessment of Toxicity

Assays were carried out in 96-well microplates. After 72 hours of treatment, the cells were washed once with 100 μL DPBS (no calcium, no magnesium). Then 100 μl of PrestoBlue solution (Life Technologies) diluted at 10% in DPBS was added to each well. Plates were incubated at 37° C. for 30 minutes. The fluorescence intensity was measured by multiwell plate reader (Glomax microplate reader, Promega, Charbonnières les Bains, France) using green filter (Excitation 525 nm/emission 580-640 nm). Fluorescence intensities values were pasted in Prism Software (Graph Pad, San Diego, CA) to perform a dose response analysis. CV25 correspond to drug concentrations at which HGPS fibroblast cell viability was 25%. CV75 correspond to drug concentrations at which HGPS fibroblast cell viability was 75%.

### Protein Extraction

Total fibroblast proteins were extracted after 72 hours treatment in 100 μL of NP40 (Invitrogen) with 1× protease and phosphatase inhibitor cocktail (Life Technologies). Lysats were incubated on ice for 30 minutes, with vortexing at 10-minute intervals. Finally, they were sonicated four times (20 sec each) and then centrifuged at 13000 rpm for 10 minutes at 4° C.

Protein concentrations were determined with the BCA™ Protein Assay (Life Technologies).

### Western Blot

Protein lysates were separated on Nupage Novex 4-12% Bis-Tris Midi precast gels (Life Technologies) and transferred to Immobilon-FL PVDF membranes (Millipore, Molsheim, France). Membranes were blocked for one hour in 1:1 diluted blocking buffer for near infrared fluorescent western blotting (Rockland, Le Perray, France). Blocked membranes were incubated with primary antibodies overnight at 4° C., following which they were washed and incubated with IR-Dye conjugated secondary antibodies for one hour at RT. Bound antibodies were detected and analyzed on an Odyssey imaging system (Li-COR Biosciences, Bad Homburg, Germany) according to the manufacturer's instructions. Revert Protein Stain (Li-COR Biosciences) was used as a total protein loading control in addition to two traditional protein loading control GAPDH and Actin. Progerin and SRSF1 levels were quantified and normalized by Revert staining using Image Studio Lite™ software developed by Li-COR.

### Antibodies

The following antibodies were used in this study: rabbit monoclonal anti-lamin A/C (ab108922, 1/1000, Abcam, Amsterdam, Netherlands), rabbit monoclonal anti-SRSF1 (SF2) (ab129108, 1/1000, Abcam), mouse monoclonal anti-GAPDH (MAB374, 1/40000, Millipore, Molsheim, France) and mouse monoclonal anti-actin (MAB1501R, 1/10000, Millipore. Secondary antibodies conjugated with IR-Dye 800CW or 680 were used according to the manufacturer's instructions (926-32213 and 926-68072, 1/5000, Li-COR Biosciences).

### Results

Mean decrease of progerin expression (presented as a percentage of the concentration measured in DMSO control) with compounds tested at CV25, in HGPS cells at T=72 hours, determined by western blot analysis and normalised to the total amount of protein (Revert protein stain) (mean of n=3 (range)) is presented in the following Table 1:

| | | Mean decrease of progerin |
|---|---|---|
| | CV25 | expression relative |
| Compound | (μM) | to DMSO control (%) |
| 1 (control) | 0.270 | 67* |
| | | (45-79) |
| 3 | 1.800 | 71 |
| | | (63-81) |
| 4 | 0.402 | 70 |
| | | (49-82) |
| 6 | 0.142 | 69 |
| | | (60-82) |
| 7 | 5.5 | 79 |
| | | (66-98) |
| 8 | 0.152 | 47 |
| | | (38-60) |
| 12 | 0.063 | 61** |
| | | (49-74) |
| 13 | 0.020 | 68*** |
| | | (44-92) |
| 14 | 0.589 | 66 |
| | | (54-73) |
| 16 | 1.750 | 66 |
| | | (47-83) |

*n = 7
**n = 2
***n = 4

Mean change (decrease (−) or increase) of progerin expression (presented as a percentage of the concentration measured in DMSO control) with compounds tested at CV75, in HGPS cells at T=72 hours, determined by western blot analysis and normalised to the total amount of protein (Revert protein stain) (mean of n=3 (range)) is presented in the following Table 2:

| | | Mean change of progerin |
|---|---|---|
| | CV75 | expression relative |
| Compound | (μM) | to DMSO control (%) |
| 1 (control) | 0.137 | −3* |
| | | (−42; 10) |
| 3 | 0.720 | 0 |
| | | (−19; 16) |
| 4 | 0.133 | −12 |
| | | (−37; 5) |
| 6 | 0.060 | 6 |
| | | (−49; 36) |
| 7 | 2.1 | −36 |
| | | (−65; −12) |
| 8 | 0.073 | −16 |
| | | (−35; 2) |
| 12 | 0.001 | −1** |
| | | (−21; 20) |
| 13 | 0.007 | −29*** |
| | | (−50; −18) |
| 14 | 0.155 | −1 |
| | | (−22; 26) |
| 16 | 0.195 | 0 |
| | | (−26; 17) |

*n = 7
**n = 2
***n = 4

It can be seen that the compounds according to the invention have a potent anti-progerin activity which is indicative of a therapeutic effect for progeria.

1. A compound of formula (I): 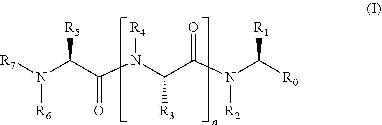
wherein:
n represents: 0, 1 or 2;
R₀ represents an aldehyde group or a protected aldehyde group;
R₂, R₄, and R₆, identical or different, with the proviso that when n=2 the two R₄ groups may be identical or different, represent: H; an alkyl group having from 1 to 6 carbon atoms, optionally substituted by one or more amino groups or carboxylic acid groups; or an alkaryl or aryl group having from 5 to 10 carbon atoms, optionally substituted by one or more amino groups, hydroxyl groups or carboxylic acid groups;
R₁, R₃ and R₅, identical or different, with the proviso that when n=2 the two R₃ groups may be identical or different, represent: H, an Arginine (Arg, R) functional group, a Leucine (Leu, L) functional group, a Norleucine (Nle) functional group, a Methionine (Met, M) functional group, a Phenylalanine (Phe, F) functional group, a Valine (Val, V) functional group, a Norvaline (Nva) functional group, or a Tyrosine (Tyr, Y) functional group; and
R₇ represents:
a protecting group, or
a group of formula (II): 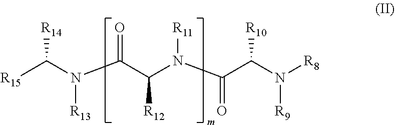
wherein:
m represents: 0, 1 or 2;
R₉, R₁₁ and R₁₃, identical or different, with the proviso that when n=2 the two R₁₁ groups may be identical or different, represent: H; an alkyl group having from 1 to 6 carbon atoms, optionally substituted by one or more amino group or carboxylic acid group; or an alkaryl or aryl group having from 5 to 10 carbon atoms, optionally substituted by one or more amino groups, hydroxyl groups or carboxylic acid groups;
R₁₀, R₁₂ and R₁₄, identical or different, with the proviso that when n=2 the two R₁₂ groups may be identical or different, represent: H, an Arginine (Arg, R) functional group, a Leucine (Leu, L) functional group, a Norleucine (Nle) functional group, a Methionine (Met, M) functional group, a Phenylalanine (Phe, F) functional group, a Valine (Val, V) functional group, a Norvaline (Nva) functional group, or a Tyrosine (Tyr, Y) functional group;
R₈ represents a linking moiety; and
R₁₅ represents an aldehyde group or a protected aldehyde group, provided that when n=1, R₀ is an aldehyde group, R₂, R₄ and R₆ all represent H, R₇ is a Z protecting group, and R₃ and R₅ both represent a leucine functional group, then R₁ does not represent a Leucine, a Norvaline or a Phenylalanine functional group,
or a pharmaceutically acceptable salt thereof.

2. The compound of formula (I) according to claim 1, wherein the compound is selected from the group consisting of: 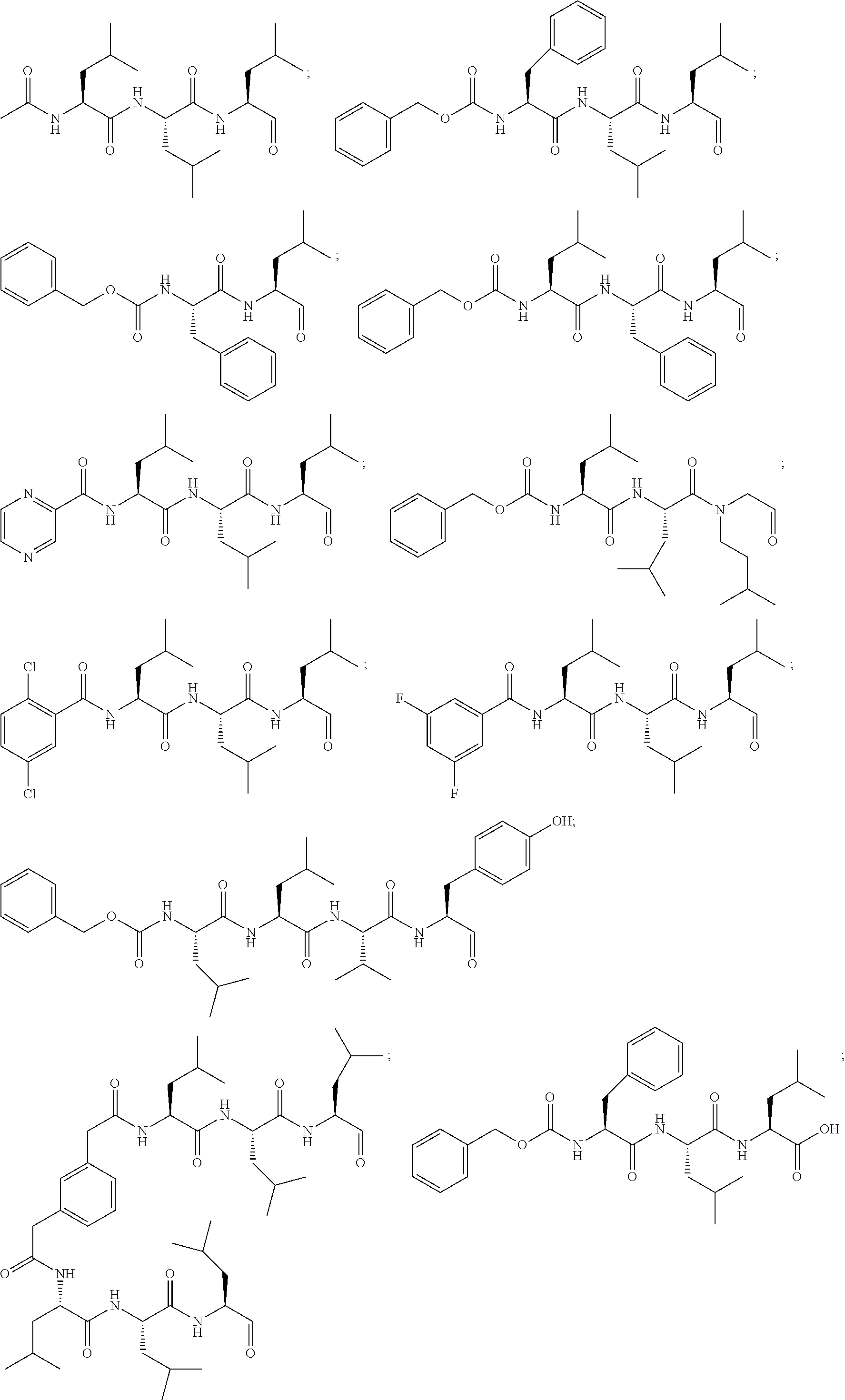 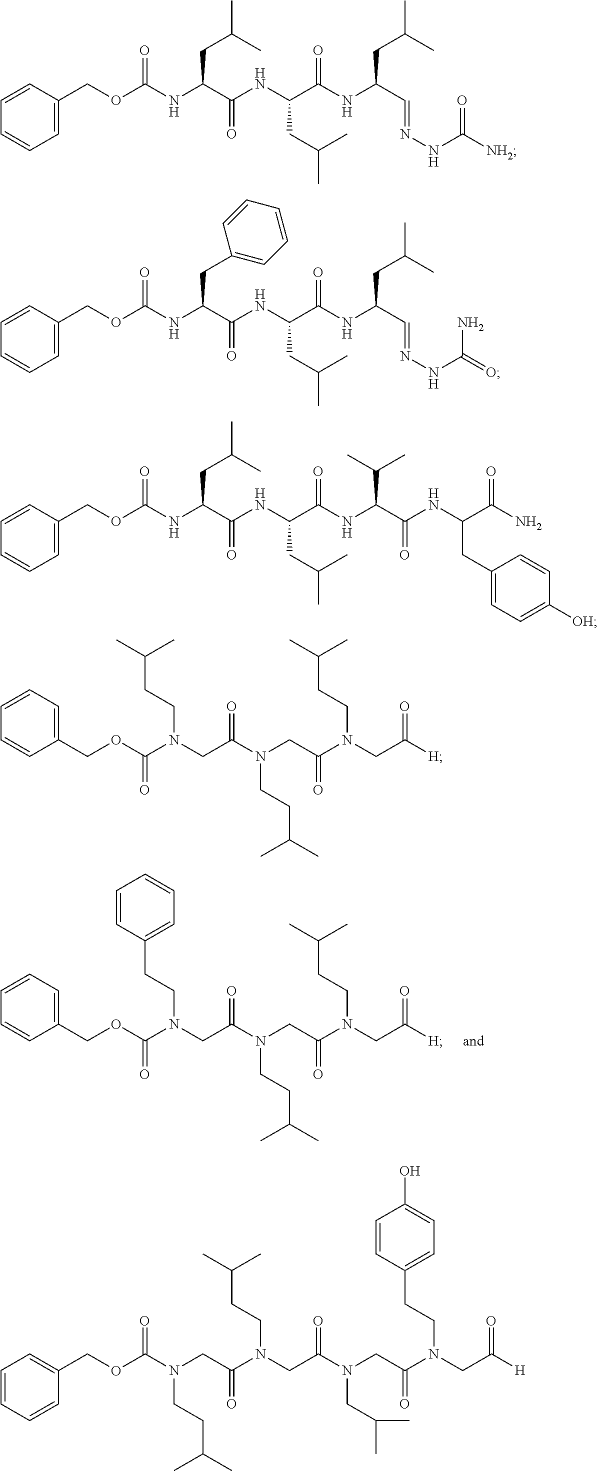
or a pharmaceutically acceptable salt thereof.

3. A method for preventing or treating a disease in an individual, comprising administering the compound of formula (I), or pharmaceutically acceptable salt thereof, according to claim 1, to the individual.

4. The method according to claim 3, wherein the method decreases the individual's cellular concentration of progerin or of prelamin A.

5. The method according to claim 3, wherein the disease is a segmental progeroid syndrome.

6. The method according to claim 3, wherein the disease is progeria.

7. The method according to claim 3, wherein the disease is Hutchinson-Gilford Progeria Syndrome (HGPS).

8. A pharmaceutical composition comprising a compound of formula (I) according to claim 1, or a pharmaceutically acceptable salt thereof, optionally in association with a pharmaceutically acceptable carrier or excipient.

9. A method for preventing or treating a disease in an individual, comprising administering the pharmaceutical composition according to claim 8, to the individual.

10. The method according to claim 9, wherein the disease is associated to progerin or to prelamin A.

11. The method according to claim 9, wherein the method decreases the individual's cellular concentration of progerin or of prelamin A.

12. The method according to claim 9, wherein the disease is a segmental progeroid syndrome.

13. The method according to claim 9, wherein the disease is progeria.

14. The method according to claim 9, wherein the disease is Hutchinson-Gilford Progeria Syndrome (HGPS).

15. The method according to claim 3, wherein the disease is associated to progerin or to prelamin A.

16. The method according to claim 6, wherein the disease is Hutchinson-Gilford Progeria Syndrome (HGPS), an HGPS-like syndrome, restrictive dermopathy, mandibuloacral dysplasia type B, an atypical progeroid syndrome, an atypical Werner syndrome or Néstor-Guillermo progeria syndrome.

17. The method according to claim 9, wherein the disease is Hutchinson-Gilford Progeria Syndrome (HGPS), an HGPS-like syndrome, restrictive dermopathy, mandibuloacral dysplasia type B, an atypical progeroid syndrome, an atypical Werner syndrome or Néstor-Guillermo progeria syndrome.
